(19) United States
(12) Patent Application Publication
(10) Pub. No.: US 2016/0030475 A1
(43) Pub. Date: Feb. 4, 2016

(54) USE OF A CLAY PRODUCT OR A CLAY BLEND PRODUCT TO DECREASE THE EFFECTS OF BACTERIAL DISEASE IN SHRIMP

(71) Applicant: Oil-Dri Corporation of America, Chicago, IL (US)

(72) Inventors: San CHING, Chicago, IL (US); Fang CHI, Chicago, IL (US); Ron L. CRAVENS, Chicago, IL (US)

(21) Appl. No.: 14/815,811

(22) Filed: Jul. 31, 2015

Related U.S. Application Data
(60) Provisional application No. 62/032,238, filed on Aug. 1, 2014.

(30) Foreign Application Priority Data
Jul. 31, 2015 (US) PCT/US15/43302

(51) Int. Cl. A61K 33/12; A61K 31/198; A61K 36/064

(52) U.S. Cl. CPC A61K 33/12; A61K 36/064; A61K 31/198

(57) ABSTRACT

Applicants have examined two Clostridium species, Clostridium difficile and Clostridium perfringens, and found that clays can adsorb the toxin produced by both and that a clay blended product can decrease the effects of a prominent in chickens and known as Necrotic Enteritis, that is caused by C. perfringens. Recently a clay or a clay blend that may be a combination of clay, yeast, and a form of a functional amino acid, was examined and found to help decrease the effects of acute hepatopancreatic necrosis disease (AHPND), which is also known as Early Mortality Syndrome (EMS) in shrimp when a challenge model that included Vibrio parahaemolyticus was used.

### RELATED APPLICATIONS AND INCORPORATION BY REFERENCE

This application is a benefit of and priority to U.S. provisional patent application Ser. No. 62/032,238 filed Aug. 1, 2014.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to a method of administering clay or a clay blend to shrimp to decrease the effects of early mortality syndrome (EMS)/acute hepatopancreatic necrosis disease (AHPND).

### BACKGROUND OF THE INVENTION

Vibrio is a bacterial genus that secrets toxins causing disease in poultry, animals, humans, fish and shrimp. An unknown toxin or toxins produced by Vibrio parahaemolyticus, has been shown to cause disease in shrimp.

The elusive pathogen causing early mortality syndrome (EMS), an emerging shrimp disease in Southeast Asia more technically known as acute hepatopancreatic necrosis disease (AHPND), and was also known as acute hepatopancreatic necrosis syndrome (AHPNS) was identified. EMS is caused by a bacterial agent, which is transmitted orally, colonizes the shrimp gastrointestinal tract and produces a toxin that causes tissue destruction and dysfunction of the shrimp digestive organ known as the hepatopancreas and does not affect humans.

The EMS/AHPND pathogen as a unique strain of a relatively common bacterium, Vibrio parahaemolyticus, that can be infected by a phage, which may cause it to release a potent toxin. A similar phenomenon occurs in the human disease cholera, where a phage makes the Vibrio cholerae bacterium capable of producing a toxin that causes cholera's life-threatening diarrhea. However, in the case of EMS in shrimp, the phage may or may not be necessary for the toxin to be released from the bacteria.

Research continues on the development of diagnostic tests for rapid detection of the EMS/AHPND pathogen that will enable improved management of hatcheries and ponds, and help lead to a long-term solution for the disease. It will also enable a better evaluation of risks associated with importation of frozen shrimp or other products from countries affected by EMS.

Since EMS was first reported in China in 2009, it has spread to Vietnam, Malaysia and Thailand, India, and Mexico, and now causes annual losses more than U.S. $1 billion. EMS outbreaks typically occur within the first 30 days after stocking a newly prepared shrimp pond, and mortality can exceed 70%.

Beside the diagnostic tests, there are no currently available methods to treat this disease. Importantly, it has been found in China and other countries that antibiotics are not effective against EMS. Sensitivity tests have shown the bacteria have already developed resistance to the full range of antibiotics.

Citation or identification of any document in this application is not an admission that such document is available as prior art to the present invention.

### SUMMARY OF THE INVENTION

There is a need for treating EMS/AHPND in shrimp. In several in vivo experiments Applicants have shown that clay and clay blend products help alleviate necrotic enteritis in chickens caused by bacterial toxins from C. perfringens. The present invention also relates to the use of clay and clay blend products as a diet supplement to improve growth and feed efficiency.

In shrimp EMS/AHPND is found to be induced by Vibrio parahaemolyticus. These bacteria also release toxins that damage the barrier of the gut including the hepatopancreas. While the toxins are still not identified they may apply a mechanism similar to many other gut damaging toxins such as α-toxin and NetB toxin from C. perfringens. Calibrin®-Z is a broad spectrum multi-toxin binder that can bind polar and non-polar toxins. Since Calibrin®-Z can bind the bacterial α-toxin and NetB toxin it may be able to bind the unknown bacterial toxins from V. parahaemolyticus.

This led to research that was conducted to see if a clay or clay blend product would decrease the effects of the bacterial toxin caused disease early mortality syndrome (EMS)/acute hepatopancreatic necrosis disease (AHPND) in shrimp.

The preferred embodiment of this product may be 100% Calibrin®-Z, a calcium montmorillonite clay, which has been heated to a temperature to decrease moisture and ground to a fine particle size. This processing, heating to between 400°-800° C., and/or fine grinding (average particle size of 32-36 μm) has been shown to increase the toxin binding ability of the clay across multiple fungal and bacterial toxins.

In addition to the preferred embodiment other materials capable of binding toxins may be substituted for Calibrin®-Z, such as other clays or sorbent minerals, diatomaceous earths, silicates, or these materials (including the base material for Calibrin®-Z) manufactured with other processes, including increased or decreased drying temperature or time or final moisture content, calcined materials, clays with added surfactants, or materials ground to a larger or smaller particle size may be used.

In addition to the preferred embodiment a blend of other products with a clay; such as yeast sources, or yeast fermentation products, yeast mannans, or whole yeast or components of the yeast cell (such as the yeast cell wall) or yeasts or yeast components from other species of yeast may also be used. Sources for mannan oligosaccharides, and/or beta glucans, or other major components of yeast could also be used, including but not limited to, other fiber or carbohydrate sources. Beside yeast any other sources such as grain, mushroom, and bacteria that can produce beta-glucan and mannan oligosaccharide may also be used. Sources of glutamate, or other energy generating amino acids (including but not limited to: α-ketoglutarate, glutamine, aspartate or the branch-chain amino acids) other functional amino acids or functional proteins could also be used.

The percent inclusion of the materials may increase or decrease from those in the preferred embodiment.

While the preferred embodiment has been shown to have value against EMS/AHPND induced by giving feeding diets contaminated with V. parahaemolyticus culture, it could have similar effects against other vibrio or clostridial or cocci based diseases or other enteric diseases in any shrimp or farmed fish species, or other species such as pigs, cattle, sheep, goats, horses, or humans.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 depicts protein toxins from Vibrio parahaemolyticus incubated with and without Calibrin®-Z and measured in kilodaltons (kDa) using gel electrophoresis. The heavy blue band of Tox A (without Calibrin®-Z in Lane 6) measuring 17 kilodaltons (kDa) and Tox B at 50 kDa in gel electrophoresis. Incubating the toxins with Calibrin®-Z decreased Tox A as seen in Lanes 4, 3, 2, and 1 and Tox B as seen in Lanes 2 and 1.

FIG. 2 depicts hourly records of total shrimp mortality in percent from the time of reverse gavage injection supernatants of EMS toxin alone (0:1) or with increasing ratios of Calibrin®-Z to toxin. Most of the mortality occurs in the first 4 hours after reverse gavage. No shrimp died in the first 6 hours of observation in groups with no toxin (PBS), or when Calibrin®-Z was incubated with the toxin at ratios of 500:1 or 250:1. Shrimp that were given the injection with EMS toxin alone had 100% of the shrimp die after 4 hours of observation.

### DETAILED DESCRIPTION OF THE INVENTION

Applicants have shown that clay and clay blend products help alleviate necrotic enteritis in chickens caused by bacterial toxins from C. perfringens.

The present invention also relates to the use of clay and clay blend products as a diet supplement to improve growth and feed efficiency.

The preferred embodiment of this product may be 100% Calibrin®-Z, a calcium montmorillonite clay, which has been heated to a temperature to decrease moisture and ground to a fine particle size. This processing, heating to between 400°-800° C., and/or fine grinding (average particle size of 32-36 μm) has been shown to increase the toxin binding ability of the clay across multiple fungal and bacterial toxins.

In addition to the preferred embodiment other materials capable of binding toxins may be substituted for Calibrin®-Z, such as other clays or sorbent minerals, diatomaceous earths, silicates, or these materials (including the base material for Calibrin®-Z) manufactured with other processes, including increased or decreased drying temperature or time or final moisture content, calcined materials, clays with added surfactants, or materials ground to a larger or smaller particle size may be used.

In addition to the preferred embodiment a blend of other products with a clay; such as yeast sources, or yeast fermentation products, yeast mannans, or whole yeast or components of the yeast cell (such as the yeast cell wall) or yeasts or yeast components from other species of yeast may also be used. Sources for mannan oligosaccharides, and/or beta glucans, or other major components of yeast could also be used, including but not limited to, other fiber or carbohydrate sources. Beside yeast any other sources such as grain, mushroom, and bacteria that can produce beta-glucan and mannan oligosaccharide may also be used. Sources of glutamate, or other energy generating amino acids (including but not limited to: α-ketoglutarate, glutamine, aspartate or the branch-chain amino acids) other functional amino acids or functional proteins could also be used.

The percent inclusion of the materials may increase or decrease from those in the preferred embodiment.

While the preferred embodiment has been shown to have value against EMS/AHPND induced by giving feeding diets contaminated with V. parahaemolyticus culture, it could have similar effects against other vibrio or clostridial or cocci based diseases or other enteric diseases in any shrimp or farmed fish species, or other species such as pigs, cattle, sheep, goats, horses, or humans.

The present invention encompasses anti-toxins and optionally yeasts, and MSG-like materials for treatment against EMS/AHPND. Other materials believed to be anti-toxins such as other clays or minerals, yeasts or yeast components, other sources of fiber, beta glucans, or enzymes. Other immunomodulators such as yeasts or yeast components, or other fibers, immunoglobulins, or sources of immunoglobulins, chitins, or corticosteroids. Other sources of energy to the gut mucosa such as functional proteins, glutamic acid, threonine, or sources of functional protein such as plasma, or functional peptides. As used herein, peptides may be a short chain of amino acids, such as 2-4 molecules of glutamate or combinations of different amino acids with or without glutamate

The present invention also involves adding heat at between 300 and 800 degrees C. for up to an hour to the clay.

The present invention also involves heating the clay, as it may impact the effectiveness of the clay. This may be done either statically using a muffle furnace or dynamically in a rotary kiln or flash dryer. The preferred embodiment of this product may be about 50 to 70% (w/w) of an anti-toxin, about 25 to 45% (w/w) of an immunomodulator, which may be a yeast and about 0.01 to 15% of a gut enhancer such as monosodium glutamate. The anti-toxin may be a calcium montmorillonite clay, advantageously heated to a temperature at between 100-800 degrees C., advantageously between 400-800 degrees C., to decrease moisture and ground to a fine particle size. This processing, heating to between 100-800 degrees C., and/or fine grinding (with an average particle size of approximately between 20 and 50 microns) has been shown to increase the toxin binding ability of the clay across multiple toxins.

The yeast may be a Pichia yeast product produced by a controlled fermentation process during the production of citric acid and is also known as citric acid press cake. The yeast may be a Pichia yeast. In an advantageous embodiment, the yeast is a Pichia guilliermondii yeast. Other Pichia species include, but are not limited to, P. anomala, P. farinose, P. heedii, P. kluyveri, P. membranifaciens, P. norvegensis, P. ohmeri, P. pastoris, P. methanolica and P. subpelliculosa.

Monosodium glutamate is a form of the amino acid glutamate.

In addition to the preferred embodiment other materials capable of binding toxins may be substituted for Amlan's products, such as other clays or sorbent minerals, diatomaceous earths, silicates, zeolites, attapulgites, hormites, or these materials or combinations of these materials (including the base material for Amlan products-) manufactured with other processes, including increased or decreased drying temperature or time or final moisture content, calcined materials, or materials ground to a larger or smaller particle size may be used. Besides grinding, any other method to produce smaller particle size including exfoliation to nanometer size is also contemplated for the present invention.

The clay may be heated to about 100° C., about 125° C., about 150° C., about 175° C., about 200° C., about 225° C., about 250° C., about 275° C., about 300° C., about 325° C., about 350° C., about 375° C., about 400° C., about 425° C., about 450° C., about 475° C., about 500° C., about 525° C., about 550° C., about 575° C., about 600° C., about 625° C., about 650° C., about 675° C., about 700° C., about 725° C., about 750° C., about 775° C., about 800° C., about 825° C., about 850° C., about 875° C., about 900° C., about 925° C., about 950° C., or about 1000° C. It may be heated for 1 minute up to 4 hours.

The average particle size of the clay may be as small as 10 nanometer to as large as 500 microns. The average particle size may advantageously be between 20 and 50 microns.

In some embodiments, the clay may further comprise a surfactant. The surfactant may be a soil surfactant or a surfactant containing alkoxylated polyols, humectants, alkylpolyglucoside esters, and polycarboxylates, sodium salts. Surfactants may also comprise a combination of soil surfactants and humectant complexes.

Other yeast sources, or yeast fermentation products, yeast mannans, or whole yeast or components of the yeast cell (such as the yeast cell wall) or mixtures of the same, or yeasts or yeast components from other species of yeast may also be used. Sources for mannan oligosaccharides, and/or beta glucans, or other major components of yeast could also be used, including but not limited to, other fiber or carbohydrate sources. Other sources of prebiotics or blends of prebiotics could also be used.

Other sources of glutamate, glutamic acid, or any of their salts, or other energy generating amino acids (including but not limited to: α-ketoglutarate, glutamine, aspartate or the branch-chain amino acids, L-glutamic acid or L-glutamine) other functional amino acids, functional peptides, or functional proteins, or nucleotides are also contemplated for the present invention.

The percent inclusion of the materials may increase or decrease from those in the preferred embodiment.

When the clay, yeast and glutamate mixture of the present invention is incorporated into a feed for shrimp or other animals or water or the water that shrimp or other aquatic animals are being raised this may be done in a manner known to one of skill in the art. In a preferred embodiment the clay, yeast and glutamate mixture of the invention is incorporated in a premix. The premix will preferably include the clay, yeast and glutamate mixture, a physiologically acceptable carrier and optionally a feedstuff. The premix is generally in a relatively concentrated form and is adapted to be diluted with other material such as one or more of the other carriers, vitamins and mineral supplements and feedstuff to form the final animal feed. The premix preferably includes the clay, yeast and glutamate mixture in a concentration in the range of from 0.1 to 70% by weight, preferably 0.5 to 50% by weight, more preferably about 0.25% by weight. The optimum concentration will depend on whether the treatment is preventative, for control or remedial and whether the clay, yeast and glutamate mixture of the invention is the only active or whether it is used in concomitant therapy with other materials and the specie and age or stage of life of the recipient.

In a preferred embodiment the concentrated composition of the clay, yeast and glutamate mixture is in a controlled-release form. The controlled release form will include the clay, yeast and glutamate mixture and a polymeric material for providing controlled release of the clay, yeast and glutamate mixture from the controlled-release system and is particularly useful in compositions for addition to solid feed material. As a result of the controlled release formulation the release of the clay, yeast and glutamate mixture may be delayed so as to occur mainly in the duodenum. A controlled release polymer may also minimize rejection of the composition due to taste or be used for rectal suppositories.

In this invention, the term, “controlled release system” is used in the same context as that in, and includes the same range of examples as quoted in “Controlled Drug Delivery” (Robinson & Lee, 1987). Many other pH-sensitive controlled-release systems which are known in the art (Robinson and Lee, 1987) may be substituted for the polymer of acrylic acid or copolymer of acrylamide and acrylic acid. For example, soluble and anionic, or insoluble cross-linked and anionic, cellulosic systems; or soluble and anionic, or insoluble cross-linked and anionic polymers derived from any generic acrylic acid polymer and/or its derivatives. Such cross-linked and insoluble polymers are preferred since they swell and also are less likely to be metabolized.

The invention also provides an animal feed composition comprising the clay, yeast and glutamate mixture of the invention and a feedstuff. The clay, yeast and glutamate mixture is preferably present in an amount of from 0.0001 to 25% of the total feed composition and preferably from 0.0001 to 5% of the total feed composition, more preferably about 0.25% of the total feed composition.

In another preferred embodiment, the clay, or clay, yeast and glutamate mixture of the invention may be formulated for addition to the water in which the shrimp are raised.

The clay, yeast and glutamate mixture of the invention is preferably administered in amounts of from 0.05 to 5000 mg/kg of body weight/day more preferably from 100 to 1000 mg/kg/day.

Examples of suitable inert carriers for use in compositions for administration of the clay, yeast and glutamate mixture of the invention include water, olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides, polyvinyl alcohol, partially hydrolyzed polyvinyl acetate and mixtures thereof.

Solid forms for oral or rectal administration may contain pharmaceutically or veterinarally acceptable binders, sweeteners, disintegrating agents, diluents, flavorings, coating agents, preservatives, lubricants and/or time delay agents. Suitable binders include gum acacia, gelatin, corn starch, gum tragacanth, sodium alginate, carboxymethylcellulose or polyethylene glycol. Suitable sweeteners include sucrose, lactose, glucose or flavonoid glycosides such as neohesperidine dihydrochalcone. Suitable disintegrating agents include corn starch, methylcellulose, polyvinlypyrrolidone, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavoring agents include peppermint oil, oil of wintergreen, cherry, orange or raspberry flavorings. Suitable coating agents include polymers or copolymers of acrylic acid and/or methacrylic acid and/or their esters, and/or their amides, waxes, fatty alcohols, zein, shellac or gluten. Suitable preservatives include sodium benzoate, vitamin E, .alpha.-tocopherol, ascorbic acid, methyl parabens, propyl parabens or sodium bisulphate. Suitable lubricants include magnesium stearate, stearic acid, sodium oleate, sodium chloride or talc. Suitable time delay agents include glyceryl monostearate or glyceryl distearate.

Suspensions for administration may further comprise dispersing agents and/or suspending agents. Suitable suspending agents include sodium carboxylmethylcellulose, methylcellulose, hydroxypropylmethylcellulose, poly-vinyl-pyrrolidone, sodium alginate or cetyl alcohol. Suitable dispersing agents include lecithin, polyoxyethylene esters or fatty acids such as stearic acid, polyoxyethylene sorbitol mono- or di-oleate, -stearate or -laurate, polyoxyethylene sorbitan mono- or di-oleate, -stearate or -laurate and the like.

The composition of the clay, yeast and glutamate mixture may further comprise one or more emulsifying agents. Suitable emulsifying agents include dispersing agents as exemplified above or natural gums such as gum acacia or gum tragacanth.

Compositions for administration in the method of the invention may be prepared by means known in the art for the preparation of compositions (such as in the art of veterinary and pharmaceutical compositions) including blending, grinding, homogenizing, suspending, dissolving, emulsifying, dispersing and where appropriate, mixing of the ingredients together with selected excipients, diluents, carriers and adjuvants.

For oral administration, the pharmaceutical or veterinary composition may be in the form of tablets, lozenges, pills, troches, capsules, elixirs, powders, including lyophilised powders, solutions, granules, suspensions, emulsions, syrups and tinctures. Slow-release, or delayed-release, forms may also be prepared, for example in the form of coated particles, multi-layer tablets or microgranules.

A study was conducted to examine the effects of two products on the mortality caused by EMS in shrimp. The products were products that had previously been shown to reduce the effect of the bacterial enteric disease necrotic enteritis in chickens. The products were: Product A) a 100% clay product, Calibrin®-Z (A); and Product B) was a blend of the clay, a yeast product, and monosodium glutamate (B). These products were all tested at two concentrations, 0.25% and 0.5% of the diet.

Shrimp fed a diet supplemented with a clay or a combination of clay, a yeast product, and monosodium glutamate and infected with Vibrio parahaemolyticus culture to induce EMS/AHPND showed reduced mortality. Survival at the end of the study in shrimp fed Product A at 0.5% was 95% and in those fed Product A at 0.25% was 85%. Those fed Product B at 0.5% inclusion had a 52.5% survival rate and those shrimp fed Product B at a 0.25% inclusion rate had a 50% survival rate. Survival in the group fed the control diet and not infected with V. parahaemolyticus was 92.5% while those fed the control diet and infected had a survival rate of 7.5%. Hisotological examination showed that shrimp fed diets containing Product A showed no hepatopancreas lesions.

Juvenile Penaeus (Litopenaeus) vannamei were fed one of four diets containing proprietary products supplied by Oil-Dri Corporation of America for 7 days prior to oral exposure to Vibrio parahaemolyticus (agent causing Acute Hepatopancreatic Necrosis Disease) to determine if the products would have an effect on survival.

Survival at termination of the study in the group fed Product A at a 0.5% inclusion rate was 95% and survival in the group fed Product A at a 0.25% inclusion rate was 85%. Termination survival in the group Product B at a 0.5% inclusion rate was 52.5% and survival in the group fed Product B at a 0.25% inclusion rate was 50%. Survival in the positive control group, fed a control diet, was 7.5% and survival in the negative control group, also fed the control diet, was 92.5%.

The animals utilized in this study were originally obtained from Shrimp Improvement Systems. A total of 240 SPF (specific pathogen free) P. vannamei were transferred from UAZ's West Campus SPF facility and stocked into twelve 90 L aquaria at 20 animals per tank.

Eight tanks were fed diets formulated with the products (2 tanks for each diet). Two tanks were designated as positive controls and two tanks served as negative environmental controls. The positive control tanks were fed a commercially pelleted shrimp diet (Rangen, Inc., 40% protein) and were challenged with Vibrio parahaemolyticus to ensure that the challenge method worked and as a comparison for survival. The negative control tanks were also fed the commercially pelleted control diet, but were not challenged with Vibrio parahaemolyticus. See Table 1 for complete stocking details.

All tanks were fed their respective diets at 5% bodyweight once a day for the duration of the study. All aquaria were outfitted with an oystershell filter, aeration and were covered with a plastic sheet to reduce the risk of cross contamination. The negative control tanks were kept isolated in a separate building and fed before the Vibrio parahaemolyticus challenge tanks.

Two products were used in this study. The first product was labeled as Product A and the second was labeled as Product B. The four diets included:
Product A—0.5% inclusion rate
Product A—0.25% inclusion rate
Product B—0.5% inclusion rate
Product B—0.25% inclusion rate

The test materials were combined with Rangen (Rangen Inc., 115 13 Ave. So. Buhl, Id.) 40% protein commercial shrimp premix, 40% water, and the binder carboxymethyl cellulose at 3% inclusion rate, and then cold extruded through a meat grinder. The resulting feed was dried overnight at 40° C. and then broken into an appropriately sized pellet.

Each tank was fed the appropriate diet for a total of 7 days prior to AHPND challenge. Once the AHPND study began, all tanks were checked daily for moribund or dead animals. A few moribund animals were preserved in Davidson's AFA fixative and processed for routine histology. All mortalities not preserved by fixation and all dead animals were removed from the tanks and frozen. At termination of the study, 2 live animals from each tank were preserved in Davidson's AFA fixative and all remaining animals were frozen. The study was terminated 15 days post initial exposure to AHPND.

On day 0 of the AHPND challenge portion of the study), all challenge aquaria were fed the appropriate diet (either Products A or B or the control diet) which had been soaked in a broth containing the AHPND inducing V. parahaemolyticus at an optical density of 1.57. No mortality was noted in any tank by day 1 post infection (p.i.), so all tanks were re-challenged on day 2 p.i. in the same manner as on day 0, but this time with a broth at an optical density of 1.26.

By day 7 p.i., no mortality had been noted in any tank with the exception of one of the positive control tanks, so the tanks were again re-challenged, but this time they were given two feedings in a single day. On day 8 p.i. of the AHPND challenge, all AHPND challenge aquaria were fed a commercially pelleted shrimp diet (Rangen Inc., 35% protein) which had been soaked in an AHPND-causing Vibrio parahaemolyticus broth with an optical density of 1.88. On the afternoon of day 8 p.i., all AHPND challenge aquaria were fed a second dose of the same Vibrio parahaemolyticus broth, but this time the broth was added to the respective Oil-Dri diet prior to feeding.

No dead or moribund animals were noted in the negative control tanks during the study, although a reduction in numbers was noted and the missing animals were attributed to cannibalism. At termination of the study 15 days post-infection (22 days total), a total of 20 live animals out of 20 stocked on day 0 were collected from the first tank and 17 live animals out of 20 were collected from the second tank. Combined termination survival was 92.5%, with individual survival rates of 100% and 85%, respectively. Please refer to Table 2 for complete survival data on both of the negative control tanks.

Diet 1 (Product A at a 0.5% inclusion rate): On day 9 post EMS exposure, one moribund animal was noted one of the challenge tanks fed Diet 1. No other dead or moribund animals were noted in this group of tanks, although a reduction in numbers was noted in one tank by day 13 post-infection. At termination of the study on day 15 post-infection, a total of 18 live animals were collected from the first tank and 20 live animals were collected from the second tank, resulting in survival rates of 90% and 100%, respectively. Combined survival for this group was 95%. Please refer to Tables 2 and 3 for the survival and mortality numbers for each tank.

Diet 2 (Product A at a 0.25% inclusion rate): No dead or moribund animals were noted in either tank fed Diet 2 during the study, although a reduction in numbers was noted on day 13 post-infection. The missing animals were likely weak and fully cannibalized before they could be removed from the tank. At termination of the study, 18 live animals were collected from the first tank and 16 animals were collected from the second tank resulting in tank survival rates of 90% and 80%, respectively. Combined survival for this group was 85%. Please refer to Tables 2 and 3 for the survival and mortality numbers for each tank.

Diet 3 (Product B at a 0.5% inclusion rate): The first dead and moribund animals were noted in one of the tanks fed Diet 3 on day 10 post-exposure (2 days after final exposure). Dead and moribund animals were collected from the first tank daily until day 12 p.i., when mortality ceased. No dead or moribund animals were collected from the second tank during the study, although a reduction in numbers was noted on day 13 post-infection. At termination of the study 3 live animals were collected from the first tank, resulting in a 15% survival rate and 18 live animals were collected from the second tank, resulting in a 90% survival rate. Combined termination survival for this group was 52.5%. Please refer to Tables 2 and 3 for the survival and mortality numbers for each tank.

Diet 4 (Product B at a 0.25% inclusion rate): The first dead and moribund animals were noted in both tanks fed Diet 4 on day 9 post EMS exposure. Dead animals were collected daily until mortality ceased on day 11 post-exposure. At termination of the study 4 live animals were collected from one tank, resulting in a 20% survival rate and 16 animals were collected from the second tank, resulting in an 80% survival rate. Combined termination survival for this group was 50%. Please refer to Tables 2 and 3 for the survival and mortality numbers for each tank.

AHPND Positive Control Group (Control diet): The first dead animals were noted in one of the P. vannamei AHPND positive control tanks on day 3 post-infection. Dead and moribund animals were collected daily from the first tank until mortality ceased on day 9 post-infection. The first dead animals were noted in the second tank on day 9 post-infection and continuing until day 10 when mortality ceased. At termination of the study, a total of 2 live animals were collected from the first tank, resulting in a 5% survival rate and 2 live animals were collected from the second tank, resulting in a 10% survival rate. Combined survival in the positive control group was 7.5% (see Tables 2 and 3).

Histological examination of representative moribund or surviving animals are in Table 4 which summarizes the histology findings from this case.

TABLE 1
Definition of all tanks utilized in the AHPND challenge.

| Tank | | | AHPND |
|---|---|---|---|
| Number | Tank Definition | Feed Definition | Challenge |
| Tank 1 | Negative control | Control diet | None |
| Tank 2 | Negative control | Control diet | None |
| Tank 3 | AHPND | Control diet | Yes |
| | Positive control | | |
| Tank 4 | AHPND | Control diet | Yes |
| | Positive control | | |
| Tank 5 | AHPND Challenge | Diet #1 | Yes |
| | | Product A at 0.5% | |
| Tank 6 | AHPND Challenge | Diet #1 | Yes |
| | | Product A at 0.5% | |
| Tank 7 | AHPND Challenge | Diet #2 | Yes |
| | | Product A at 0.25% | |
| Tank 8 | AHPND Challenge | Diet #2 | Yes |
| | | Product A at 0.25% | |
| Tank 9 | AHPND Challenge | Diet #3 | Yes |
| | | Product B at 0.5% | |
| Tank 10 | AHPND Challenge | Diet #3 | Yes |
| | | Product B at 0.5% | |
| Tank 11 | AHPND Challenge | Diet #4 | Yes |
| | | Product B at 0.25% | |
| Tank 12 | AHPND Challenge | Diet #4 | Yes |
| | | Product B at 0.25% | |

TABLE 2
Survival Results for AHPND Challenge

| | | | | Number | | |
|---|---|---|---|---|---|---|
| | | | # | Collected | Final | Combined |
| Tank | Oil-Dry | Tank | Stocked | Live (Day | Survival | Survival at |
| # | Diet | Definition | (Day 0) | 15 p.i.) | by Tank | Termination |
| 1 | control | Neg. Control | 20 | 20 | 100.00% | 92.5% |
| 2 | control | Neg. Control | 20 | 17 | 85.00% | |
| 3 | control | Pos. Control | 20 | 1 | 5.00% | 7.5% |
| 4 | control | Pos. Control | 20 | 2 | 10.00% | |
| 5 | Diet 1 | AHPND | 20 | 18 | 90.00% | 95.0% |
| | | Challenge | | | | |
| 6 | Diet 1 | AHPND | 20 | 20 | 100.00% | |
| | | Challenge | | | | |
| 7 | Diet 2 | AHPND | 20 | 18 | 90.00% | 85.0% |
| | | Challenge | | | | |
| 8 | Diet 2 | AHPND | 20 | 16 | 80.00% | |
| | | Challenge | | | | |
| 9 | Diet 3 | AHPND | 20 | 3 | 15.00% | 52.5% |
| | | Challenge | | | | |
| 10 | Diet 3 | AHPND | 20 | 18 | 90.00% | |
| | | Challenge | | | | |
| 11 | Diet 4 | AHPND | 20 | 4 | 20.00% | 50.0% |
| | | Challenge | | | | |
| 12 | Diet 4 | AHPND | 20 | 16 | 80.00% | |
| | | Challenge | | | | |

TABLE 3
Daily Mortality by Tank in AHPND Challenge

| | Day | Day | Day | Day | Day | Day | Day | Day | Day | Day | Day | Day | Day | Day | Total | Total | Percent |
|---|---|---|---|---|---|---|---|---|---|---|---|---|---|---|---|---|---|
| Tank/Diet # | 1 | 2 | 3 | 4 | 5 | 6 | 7 | 8 | 9 | 10 | 11 | 12 | 13 | 14 | Dead | Stocked | Collected |
| 1 Negative | 0 | 0 | 0 | 0 | 0 | 0 | 0 | 0 | 0 | 0 | 0 | 0 | 0 | 0 | 0 | 20 | 0.00% |
| Control | | | | | | | | | | | | | | | | | |
| 2 Negative | 0 | 0 | 0 | 0 | 0 | 0 | 0 | 0 | 0 | 0 | 0 | 0 | 0 | 0 | 0 | 20 | 0.00% |
| Control | | | | | | | | | | | | | | | | | |
| 3 Positive | 0 | 0 | 1 | 6 | 2 | 4 | 2 | 0 | 1 | 0 | 0 | 0 | 0 | 0 | 16 | 20 | 80.00% |
| Control | | | | | | | | | | | | | | | | | |
| 4 Positive | 0 | 0 | 0 | 0 | 0 | 0 | 0 | 0 | 11 | 5 | 0 | 0 | 0 | 0 | 16 | 20 | 80.00% |
| Control | | | | | | | | | | | | | | | | | |
| 5 AHPND | 0 | 0 | 0 | 0 | 0 | 0 | 0 | 0 | 1 | 0 | 0 | 0 | 0 | 0 | 1 | 20 | 5.00% |
| Diet 1 | | | | | | | | | | | | | | | | | |
| 6 AHPND | 0 | 0 | 0 | 0 | 0 | 0 | 0 | 0 | 0 | 0 | 0 | 0 | 0 | 0 | 0 | 20 | 0.00% |
| Diet 1 | | | | | | | | | | | | | | | | | |
| 7 AHPND | 0 | 0 | 0 | 0 | 0 | 0 | 0 | 0 | 0 | 0 | 0 | 0 | 0 | 0 | 0 | 20 | 0.00% |
| Diet 2 | | | | | | | | | | | | | | | | | |
| 8 AHPND | 0 | 0 | 0 | 0 | 0 | 0 | 0 | 0 | 0 | 0 | 0 | 0 | 0 | 0 | 0 | 20 | 0.00% |
| Diet 2 | | | | | | | | | | | | | | | | | |
| 9 AHPND | 0 | 0 | 0 | 0 | 0 | 0 | 0 | 0 | 0 | 5 | 6 | 1 | 0 | 0 | 12 | 20 | 60.00% |
| Diet 3 | | | | | | | | | | | | | | | | | |
| 10 AHPND | 0 | 0 | 0 | 0 | 0 | 0 | 0 | 0 | 0 | 0 | 0 | 0 | 0 | 0 | 0 | 20 | 0.00% |
| Diet 3 | | | | | | | | | | | | | | | | | |
| 11 AHPND | 0 | 0 | 0 | 0 | 0 | 0 | 0 | 0 | 1 | 9 | 3 | 0 | 0 | 1 | 14 | 20 | 70.00% |
| Diet 4 | | | | | | | | | | | | | | | | | |
| 12 AHPND | 0 | 0 | 0 | 0 | 0 | 0 | 0 | 0 | 1 | 1 | 0 | 0 | 0 | 0 | 2 | 20 | 10.00% |
| Diet 4 | | | | | | | | | | | | | | | | | |

Note:
Day 1 = One day after introducing V. parahaemolyticus to all challenge tanks.

TABLE 4
Summary of histological findings from AHPND challenge

| | Number of | | |
|---|---|---|---|
| | shrimp | AHPND/ | |
| Tank # Treatment | examined | EMS(1) | Comments |
| Tank 1 Control | 2 | Not | Survivors |
| Diet | | detected | |
| Tank 3 | 1 | G2 | Moribund |
| Positive Control | 1 | G2 | Moribund |
| Control Diet | | | |
| Tank 4 | 2 | Not | Survivors |
| Positive Control | | detected | |
| Control Diet | | | |
| Tank 5 | 1 | Not | Moribund |
| EMS | | detected | (likely in |
| Diet #1 | | | molt) |
| Tank 6 | 2 | Not | Survivors |
| EMS | | detected | |
| Diet #1 | | | |
| Tank 7 | 2 | Not | Survivors |
| EMS | | detected | |
| Diet #2 | | | |
| Tank 8 | 1 | Not | Survivor |
| EMS | | detected | |
| Diet #2 | 1 | Not | Survivor |
| | | detected | |
| Tank 9 | 1 | G3 | Survivor |
| EMS | | | |
| Diet #3 | | | |
| Tank 10 | 2 | Not | Survivors |
| EMS | | detected | |
| Diet #3 | | | |
| Tank 11 | 1 | G4 | Fresh mort |
| EMS | | | |
| Diet #4 | | | |
| Tank 12 | 2 | Not | Survivors |
| EMS | | detected | |
| Diet #4 | | | |

NOTES:
(1)AHPND/EMS = Lesions diagnostic of Acute hepatopancreatic necrosis diseases, also known as Early mortality syndrome.
2) G-trace to G4 = Severity grade of infection/lesion, according to enclosed severity grade table. Numbers on the left side of the parentheses indicate number of shrimp affected.

### APPENDIX 1

| Severity | |
|---|---|
| Grade | Clinical Findings |
| 0 | No signs of infection/infestation by pathogen, parasite, |
| | or epicommensal present. |
| trace | Signs of infection/infestation by pathogen, parasite or |
| | epicommensal are present at just above diagnostic procedure |
| | minimum detection limits. |
| 1 | Signs of infection/infestation by pathogen, parasite or |
| | epicommensal are present, but at levels that may be below |
| | those needed for clinical disease. |
| | Agent detected may be in early stages of infection and |
| | represent preclinical disease. |
| 2 | Moderate signs of infection/infestation as shown by low to |
| | moderate numbers of parasite or epicommensal, or by number |
| | and severity of pathogen caused lesions. |
| | Prognosis is for possible production losses and/or slight |
| | increases in mortality if no treatment (if treatable) or |
| | management change is applied. |
| 3 | Moderate to high signs of disease apparent as shown by |
| | relatively higher numbers of parasite or epicommensal, or |
| | by number and severity of pathogen caused lesions. |
| | Potentially lethal prognosis if no treatment (if treatable) |
| | or management change is applied. |
| 4 | High numbers of parasite or epicommensal present, or for |
| | pathogen caused infections the presence of severe lesions |
| | and advanced tissue destruction. |
| | Lethal prognosis, especially under conditions conducive to |
| | disease development (i.e. with low oxygen, ecdysis, changes |
| | in salinity or temperature, etc.) |

The study was repeated using Calibrin®-Z since it had previously shown to have the most beneficial effect on mortality caused by early mortality syndrome (EMS) or acute hepatopancreatic necrosis disease (AHPND) in shrimp in the previous study. This product was tested at two concentrations, 0.25% and 0.5% of the diet.

Shrimp fed a diet supplemented with Calibrin®-Z and infected with Vibrio parahaemolyticus culture to induce EMS/AHPND showed reduced mortality compared to a challenged group that was not fed Calibrin®-Z. Survival at the termination of the study in the group fed Calibrin®-Z at a 0.25% inclusion rate was 66.7% and survival in the group fed Calibrin®-Z at a 0.5% inclusion rate was 39.8%. Survival in the positive control group, the group challenged with V. parahaemolyticus and fed the control diet, was 1.5% and survival in the negative (unchallenged) control group, also fed the control diet, was 100%. Histological examination showed that shrimp fed diets containing Calibrin®-Z showed no hepatopancreas lesions.

Juvenile Penaeus (Litopenaeus) vannamei were fed one of two diets (0.25 or 0.5% inclusion) containing a proprietary product supplied by Oil-Dri Corporation of America for 7 days prior to oral exposure to Vibrio parahaemolyticus (agent causing AHPND) to determine if the product would have an effect on survival.

Survival at the end of the study in the group fed Calibrin®-Z at a 0.25% inclusion rate was 66.7% and those fed Calibrin®-Z at a 0.5% inclusion rate had a 39.8% survival rate. Survival in the positive control group, fed a control diet, was 1.5% and survival in the negative control group (the only unchallenged group), also fed the control diet, was 100%.

The animals utilized in this study were originally obtained from Shrimp Improvement Systems. A total of 264 SPF (specific pathogen free) P. vannamei were transferred from University of Arizona's West Campus SPF facility and stocked into twelve 90 L aquaria at 22 animals per tank.

Six tanks were fed diets formulated with the product (3 tanks for each diet). Three tanks were designated as positive controls and three tanks served as negative environmental controls. The positive control tanks were fed a commercially pelleted shrimp diet and were challenged with Vibrio parahaemolyticus to ensure that the challenge method worked and as a comparison for survival. The negative control tanks were also fed the commercially pelleted control diet, but were not challenged with Vibrio parahaemolyticus. One negative control tank was set up next to the AHPND challenge tanks and the two remaining tanks were kept isolated in a separate building. See Table 5 for complete stocking details.

All tanks were fed their respective diets at 5% bodyweight once a day for the duration of the study. All aquaria were outfitted with an oyster-shell filter, aeration and were covered with a plastic sheet to reduce the risk of cross contamination. All negative control tanks were kept isolated in a separate building and fed before the Vibrio parahaemolyticus challenge tanks.

Product Calibrin®-Z was used in this study. The three diets made included: Diet 1—Calibrin®-Z at 0.25% inclusion rate, Diet 2—Calibrin®-Z at 0.50% inclusion rate and Diet 3—Control Diet (no Calibrin® Z).

The test materials were combined with Rangen (Rangen Inc., 115 13 Ave. So. Buhl, Id.) 40% protein commercial shrimp premix, 40% water, and the binder carboxymethyl cellulose at 3% inclusion rate, and then cold extruded through a meat grinder. The resulting feed was dried overnight at 40° C. and then broken into an appropriately sized pellet.

Each tank was fed the appropriate diet for a total of 7 days prior to AHPND challenge. Once the AHPND study began, all tanks were checked daily for moribund or dead animals. A few moribund animals were preserved in Davidson's AFA fixative and processed for routine histology. All mortalities not preserved by fixation and all dead animals were removed from the tanks and frozen. At termination of the study, 1-2 live survivors from each tank were preserved in Davidson's AFA fixative and any remaining animals were frozen. The study was terminated 7 days post initial exposure to AHPND.

Different from the previous study, an 18-20 hour culture of Vibrio parahaemolyticus which causes AHPND (Vietnam isolate) with a reduced optical density of 1.5 was utilized in this study. On day 0 of the AHPND challenge portion of the study, all challenge aquaria were fed a commercially pelleted shrimp diet (Rangen Inc., 40% protein) which had been soaked in a broth containing the AHPND inducing V. parahaemolyticus at an optical density of 0.071 with 5.0×10⁵ colony forming units. On the afternoon of day 0 post infection all AHPND challenge aquaria were fed a second dose of the same Vibrio parahaemolyticus broth, but this time the broth was added to the respective diets prior to feeding. That same day, all animals in each tank were counted to obtain an actual day 0 start number for the AHPND challenge (see Table 6).

No mortality was noted in any of the challenge tanks by day 2 post infection, so all tanks were re-challenged on day 3 post-infection in the same manner as on day 0, but this time with a broth at an optical density of 1.75 with 1.1×10⁹ colony forming units.

No dead or moribund animals were noted in the negative control tanks during the study. At termination of the study 10 days post-infection, a total of 21 live animals out of 21 counted on day 0 post-infection were collected from all three negative control tanks, resulting in individual and combined survival rates of 100%. Please refer to Table 6 for complete survival data on all three negative control tanks.

Diet 1 (Calibrin® Z at a 0.25% inclusion rate): The first dead animals were noted in one of the challenge tanks fed Diet 1 beginning on day 4 post initial challenge. Dead and moribund animals were collected from that tank daily until the last animal died on day 6 post infection. No dead or moribund animals were noted in the remaining two tanks during the challenge study. At termination of the study on day 10 post-infection, no survivors were collected from the first tank and 19 and 20 survivors were collected from the remaining two tanks, resulting in survival rates of 0% and 100%, respectively. Combined survival for this group was 66.7%. Please refer to Tables 2 and 3 for the survival and mortality numbers for each tank.

Diet 2 (Calibrin® Z at a 0.5% inclusion rate): The first dead and moribund animals were noted in the two tanks fed Diet 2 beginning on day 3 post-infection. Dead and moribund animals were collected daily from those two tanks until mortality ceased on day 7 post-infection.

No dead or moribund animals were noted in the final tank in this group during the challenge study. At termination of the study, 3 live animals were collected from the first tank, 1 animal was collected from the second tank and 20 live animals were collected from the final tank. Tank survival rates were 14%, 5% and 100%, respectively. Combined survival for this group was 39.8%. Please refer to Tables 6 and 7 for the survival and mortality numbers for each tank. AHPND Challenge Group (Control diet): The first dead animals were noted in one of the AHPND positive control tanks on day 3 post-infection. The first dead animals were noted in the second tank on day 4 post-infection and in the third tank on day 5 post-infection. Dead and moribund animals were collected daily from all three tanks until mortality ceased on day 8 post-infection. At termination of the study, no live animals were collected from two tanks and 1 live animal was collected from the last tank, resulting in a 0% survival rates for two tanks and a 4% survival rate for the last tank. Combined survival in the positive control group was 1.5% (see Tables 6 and 7).

Histological examinations of representative moribund or surviving animals are in Table 8 which summarizes the histology findings from this case.

TABLE 5
Definition of all tanks utilized in
the Amlan Industries AHPND challenge.

| | Tank | Feed | AHPND |
|---|---|---|---|
| Tank Number | Definition | Definition | Challenge |
| Tank 1 | Negative control | Control diet | None |
| | SPF room | | |
| Tank 2 | Negative control | Control diet | None |
| | SPF room | | |
| Tank 3 | Negative control | Control diet | None |
| | AHPND room | | |
| Tank 4 | AHPND | Control diet | Yes |
| | Positive control | | |
| Tank 5 | AHPND | Control diet | Yes |
| | Positive control | | |
| Tank 6 | AHPND | Control diet | Yes |
| | Positive control | | |
| Tank 7 | AHPND Challenge | Diet #1 | Yes |
| | | Calibrin ®-Z at 0.25% | |
| Tank 8 | AHPND Challenge | Diet #1 | Yes |
| | | Calibrin ®-Z at 0.25% | |
| Tank 9 | AHPND Challenge | Diet #1 | Yes |
| | | Calibrin ®-Z at 0.25% | |
| Tank 10 | AHPND Challenge | Diet #2 | Yes |
| | | Calibrin ®-Z at 0.50% | |
| Tank 11 | AHPND Challenge | Diet #2 | Yes |
| | | Calibrin ®-Z at 0.50% | |
| Tank 12 | AHPND Challenge | Diet #2 | Yes |
| | | Calibrin ®-Z at 0.50% | |

TABLE 6
Survival Results for Amlan Industries AHPND Challenge.

| | | | | | | | Combined % |
|---|---|---|---|---|---|---|---|
| | | | | | Number | | Survival at |
| | | | Number | Number | Collected | Final | Termination |
| Tank | Amlan | Tank | Stocked | Counted | Live | Survival | (day 0 p.i. to |
| Number | Diet | Definition | (Day 0) | (Day 0 p.i.) | (Day 10 p.i.) | by Tank | day 10) |
| 1 | Control | Neg. Control | 22 | 21 | 21 | 100.00% | 100.00% |
| 2 | Control | Neg. Control | 22 | 21 | 21 | 100.00% | |
| 3* | Control | Neg. Control | 22 | 21 | 21 | 100.00% | |
| 4 | Control | Pos. Control | 22 | 22 | 1 | 4.55% | 1.5% |
| 5 | Control | Pos. Control | 22 | 22 | 0 | 0.00% | |
| 6 | Control | Pos. Control | 22 | 20 | 0 | 0.00% | |
| 7 | Diet 1 | AHPND Challenge | 22 | 19 | 0 | 0.00% | 66.7% |
| 8 | Diet 1 | AHPND Challenge | 22 | 19 | 19 | 100.00% | |
| 9 | Diet 1 | AHPND Challenge | 22 | 20 | 20 | 100.00% | |
| 10 | Diet 2 | AHPND Challenge | 22 | 21 | 3 | 14.29% | 39.8% |
| 11 | Diet 2 | AHPND Challenge | 22 | 20 | 1 | 5.00% | |
| 12 | Diet 2 | AHPND Challenge | 22 | 20 | 20 | 100.00% | |

*This negative control tank was set up right next to the AHPND challenge tanks.

TABLE 7
Daily Mortality by Tank in Amlan Industries AHPND Challenge (UAZ case 14-170).

| | | | | | | | | | | | Total | Total | |
|---|---|---|---|---|---|---|---|---|---|---|---|---|---|
| | Day | Day | Day | Day | Day | Day | Day | Day | Day | Day | Dead | Number | Percent |
| Tank/Diet # | 1 | 2 | 3 | 4 | 5 | 6 | 7 | 8 | 9 | 10 | Collected | Stocked | Collected |
| 1 Negative Control | 0 | 0 | 0 | 0 | 0 | 0 | 0 | 0 | 0 | 0 | 0 | 21 | 0.00% |
| 2 Negative Control | 0 | 0 | 0 | 0 | 0 | 0 | 0 | 0 | 0 | 0 | 0 | 21 | 0.00% |
| 3 Negative Control | 0 | 0 | 0 | 0 | 0 | 0 | 0 | 0 | 0 | 0 | 0 | 21 | 0.00% |
| 4 Positive Control | 0 | 0 | 9 | 9 | 2 | 1 | 0 | 0 | 0 | 0 | 21 | 22 | 95.45% |
| 5 Positive Control | 0 | 0 | 0 | 8 | 13 | 0 | 0 | 0 | 1 | 0 | 22 | 22 | 100.00% |
| 6 Positive Control | 0 | 0 | 0 | 0 | 16 | 2 | 1 | 1 | 0 | 0 | 20 | 20 | 100.00% |
| 7 AHPND Diet 1 | 0 | 0 | 0 | 8 | 10 | 1 | 0 | 0 | 0 | 0 | 19 | 19 | 100.00% |
| 8 AHPND Diet 1 | 0 | 0 | 0 | 0 | 0 | 0 | 0 | 0 | 0 | 0 | 0 | 19 | 0.00% |
| 9 AHPND Diet 1 | 0 | 0 | 0 | 0 | 0 | 0 | 0 | 0 | 0 | 0 | 0 | 20 | 0.00% |
| 10 AHPND Diet 2 | 0 | 0 | 11 | 6 | 1 | 0 | 0 | 0 | 0 | 0 | 18 | 21 | 85.71% |
| 11 AHPND Diet 2 | 0 | 0 | 11 | 1 | 3 | 1 | 2 | 0 | 0 | 0 | 18 | 20 | 90.00% |
| 12 AHPND Diet 2 | 0 | 0 | 0 | 0 | 0 | 0 | 0 | 0 | 0 | 0 | 0 | 20 | 0.00% |

Note:
Day 1 is one day after introducing V. parahaemolyticus at 106 dose and day 3 is when all tanks were re-challenged with 108 dose

TABLE 8
Summary of histological findings from Amlan AHPND challenge.

| | | Number of | | |
|---|---|---|---|---|
| Tank # | Sampling Date | shrimp | AHPND/ | |
| Treatment | (mm/dd/yy) | examined | EMS(1) | Comments |
| Tank 1 | May 13, 2014 | 2 | Not Detected | Healthy |
| Negative | Day 0 | | | |
| Control | | | | |
| Tank 3 | May 30, 2014 | 2 | Not Detected | Survivors |
| Negative | Day 10 p.i.(2) | | | |
| Control | | | | |
| Tank 4 | May 23, 2014 | 1 | G4(3) | Moribund |
| Positive | Day 3 p.i. | | | |
| Control | May 30, 2014 | 1 | Not Detected | Survivor |
| | Day 10 p.i. | | | |
| Tank 6 | May 26, 2014 | 1 | G4 | Recently |
| | | | | dead |
| Positive | Day 6 p.i. | | | |
| Control | | | | |
| Tank 8 | May 30, 2014 | 2 | Not Detected | Survivors |
| EMS Diet 1 | Day 10 p.i. | | | |
| Tank 9 | May 30, 2014 | 2 | Not Detected | Survivors |
| EMS Diet 1 | Day 10 p.i. | | | |
| Tank 10 | May 23, 2014 | 1 | G3 | Moribund |
| EMS Diet 2 | Day 3 p.i. | | | |
| | May 30, 2014 | 1 | 1(G3) | Survivors |
| | Day 10 p.i. | | 1(Not Detected) | |
| Tank 11 | May 30, 2014 | 1 | G4 | Recently |
| | | | | dead |
| EMS Diet 2 | Day 10 p.i. | | | |
| | May 30, 2014 | 1 | Not Detected | Survivor |
| | Day 10 p.i. | | | |
| Tank 12 | May 30, 2014 | 2 | Not Detected | Survivors |
| EMS Diet 2 | Day 10 p.i. | | | |

(1)AHPND/EMS = Lesions diagnostic of Acute hepatopanereatic necrosis diseases, also known as Early mortality syndrome.
(2)p.i. = Post-infection.
(3)G-trace to G4 = Severity grade of infection/lesion, according to enclosed severity grade table. Numbers on the left side of the parentheses indicate number of shrimp.

Another study was conducted to examine the binding effects of Calibrin®-Z on the Vibrio parahaemolyticus producing toxins in vitro and the toxicity to shrimp by reverse gavage injection in vivo. Incubating Calibrin®-Z with the two toxins that cause early mortality syndrome (EMS) or acute hepatopancreatic necrosis disease (AHPND) in shrimp reduced the amount of the toxins that were free in the solution. Mortality decreased from 95% (control) to 5% (Calibrin®-Z) when Calibrin®-Z was incubated with the toxin prior to reverse gavage injection of the supernatant into shrimp.

In an in vitro study, Calibrin®-Z was incubated at different concentrations, 500, 250, 125, 62.5, and 31.25 mg with 1 mg of bacterial toxin fraction, of EMS-inducing V. parahaemolyticus. The toxin fraction was prepared from the 60% ammonium sulfate-precipitated culture broth of EMS-inducing V. parahaemolyticus, which was dialyzed before incubation with Calibrin®-Z.

After incubation with gently agitating at room temperature for 30 min, the mixed solution was centrifuged (6,000 rpm for 15 min at 4° C.) and the supernatant was collected and divided into two parts. One part of the supernatant (approximately 20 μg total protein) was used for an in vitro study looking at the amount of the toxin remaining in the supernatant using gel electrophoresis (12% SDS-PAGE) and the patterns were visualized by CBB-G250 staining. The second part (approximately 40 μg total protein) was used in an in vivo study done by performing reverse gavage injection of the supernatant into live shrimp. The supernatant of the control group was prepared the same way but without adding the Calibrin-Z.

The white shrimp used for in vivo study had a fresh weight of 2-4 gram. Ten shrimp for each treatment were used for toxin injection and the study was repeated twice. Supernatant from the in vitro study was injected via reverse gavage (with the same volume giving 10 μg of total protein per gram body weight in non-Calibrin®-Z treated supernatant). Shrimp mortality was observed hourly for the first 6 hours and then at 24 hours after injection. The moribund shrimp at the end of the experiment were collected and fixed in Davidson's fixative for histological examination to confirm that the mortality had been caused by the EMS toxin.

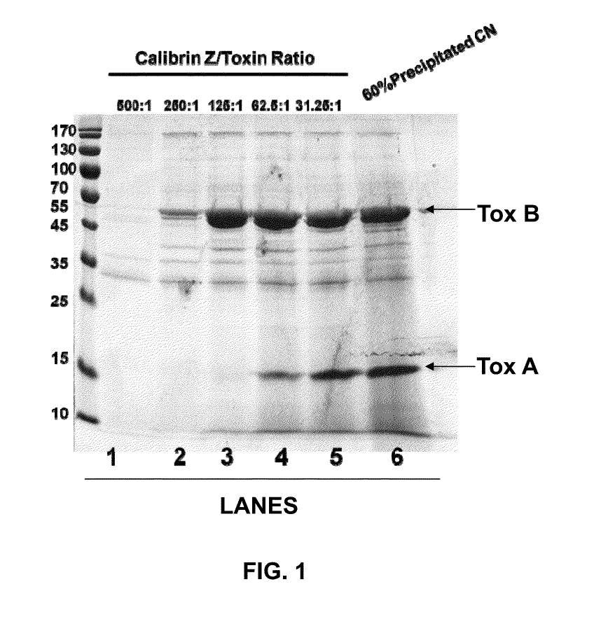

In vitro: The bacterial toxin preparation contained two toxin proteins named ToxA and ToxB. ToxA has a weight around 17 kDa (kilodaltons) while ToxB has a weight around 50 kDa. This can be seen by looking Lane 6 of FIG. 1 and comparing the blue bands that are seen there to the weights of the standards that were run, seen on the far left. The ToxA band at 17 kDa has disappeared after incubation of 1 mg of toxin with 125 mg of Calibrin®-Z (Lane 3). The reduction of ToxB and the disappearance of the ToxA band can be seen in Lane 2, which was when the toxins were incubated with 250 mg Calibrin®-Z. Both Toxin A and ToxinB bands disappeared completely when the toxin preparation was incubated with 500 mg Calibrin®-Z (Lane 1). Similar results were found when this test was repeated.

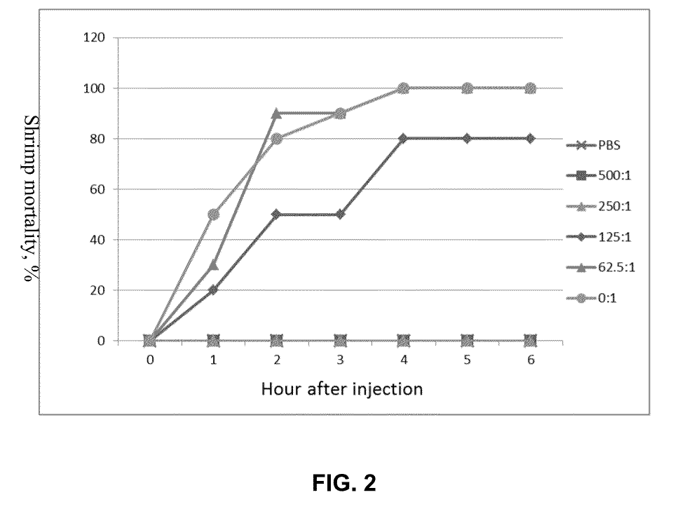

In vivo: After reverse gavage injection, shrimp mortality was recorded every hour for the first 6 hours (Exp 1) and the data is presented in FIG. 2. Mortality mainly occurred during the first 4 hours after injection. Examination of the hepatopancreas of the dead shrimp determined that EMS was the cause of death.

Treatment groups of the buffer solution with no toxin (PBS), or a Calibrin®-Z:toxin ratio of 500:1 or 250:1 had no mortality in 6 hours of observation. Calibrin®-Z:toxin at 250:1 ratio and higher fully prevented the EMS induced mortality during this time period. The mortality that was seen at 24 h post-injection for the two studies is shown in Table 1. Only the results from incubation of the toxin preparation with 125, 250, and 500 mg Calibrin®-Z and control group were expressed in the table since the lower amount of Calibrin®-Z did not show the beneficial effect. The patterns of the results are similar to those of the in vitro study. The amount of 500 mg of Calibrin®-Z was the most effective concentration among the treatments. When Calibrin®-Z was not added mortality was as high as 95%, but was only 5% when 500:1 Calibrin®-Z to toxin was added. This suggests that Calibrin®-Z can reduce the mortality that is induced by the EMS-causing bacterial toxins (ToxA and ToxB).

Bacterial toxins are a serious threat to global animal protein production in the livestock, poultry, and aquaculture industries. In these studies, Calibrin®-Z reduced the amount of the Vibrio parahaemolyticus toxins (ToxA and ToxB), which are the cause of EMS/AHPND. This was seen using both gel electrophoresis and live shrimp. In conclusion, Calibrin®-Z provides a layer of toxin control to reduce the mortality induced by EMS.

Although the present invention and its advantages have been described in detail, it should be understood that various changes, substitutions and alterations can be made herein without departing from the spirit and scope of the invention as defined in the appended claims.

The invention is further described by the following numbered paragraphs:

1. A method for treating early mortality syndrome (EMS)/acute hepatopancreatic necrosis disease (AHPND) in an animal in need thereof comprising administering clay or a clay blend to the animal, thereby treating early mortality syndrome (EMS)/acute hepatopancreatic necrosis disease (AHPND).

2. The method of claim 1 wherein the clay blend comprises a clay, a glutamate and/or a yeast.

3. The method of claim 1 wherein the clay blend comprises a calcium montmorillonite clay.

4. The method of claim 1, 2, or 3 wherein the early mortality syndrome (EMS)/acute hepatopancreatic necrosis disease (AHPND) is caused by a Vibrio species.

5. The method of claim 4 wherein the Vibrio species is Vibrio parahaemolyticus.

6. The method of any one of claims 1 to 5, wherein the animal is a shrimp, fish, clam, crab, lobster, pig, cattle, sheep, goat, horse, or human.

7. The method of any one of claims 1 to 5, wherein the animal is an aquatic species.

8. The method of claim 7, wherein the aquatic species is a shrimp or a fish.

9. The method of claim 8 wherein the fish is a farmed shrimp or a farmed fish.

10. The method of any one of claims 1-9 wherein the clay or clay blend is a modified clay or clay blend.

11. The method of any one of claims 1 to 10, wherein the clay or clay blend is administered as a diet supplement.

12. The method of any one of claims 1 to 11, wherein the clay blend is about 50 to 90% w/w of the clay, about 0.01 to 20% w/w of the yeast and about 0.01 to 20% w/w of the glutamate.

13. The method of claim 12, wherein the clay blend is about 80% w/w of the clay, about 10% w/w of the yeast and about 10% w/w of the glutamate.

14. The method of any one of claims 1 to 13, wherein the clay is a calcium montmorillonite clay.

15. The method of any one of claims 1 to 14, wherein the clay is a sorbent mineral, a diatomaceous earth, a silicate, a zeolite, an attapulgite, or a combination thereof.

16. The method of any one of claims 1 to 15, wherein the clay is heated to between about 100° C. to about 800° C.

17. The method of any one of claims 1 to 16, wherein the clay is ground or exfoliated to a particle size of about 10 nanometers to 50 microns.

18. The method of any one of claims 1-17, wherein the clay further comprises a surfactant.

19. The method of claim 18, wherein the surfactant is a soil surfactant or a surfactant containing alkoxylated polyols, humectants, alkylpolyglucoside esters, and polycarboxylates, sodium salts or a combination of soil surfactants and humectant complexes.

20. The method of any one of claims 1-19, wherein the clay further comprises an organic compound.

21. The method of any one of claims 2 to 20, wherein the yeast is a Pichia yeast.

22. The method of claim 21, wherein the yeast is a Pichia guilliermondii yeast.

23. The method of claim 21 or 22, wherein the yeast is a citric acid press cake.

24. The method of any one of claims 2 to 23, wherein the yeast is a yeast fermentation product.

25. The method of any one of claims 2 to 24, wherein the yeast is a yeast component.

26. The method of claim 25, wherein the yeast component is a yeast mannan, a yeast cell wall, a mannan oligosaccharide, a beta glucan, a fiber, a carbohydrate source, a prebiotic, or a combination thereof.

27. The method of any one of claims 2 to 27 wherein the yeast component is isolated from grain, mushroom and/or bacteria.

28. The method of any one of claims 2 to 27, wherein the yeast is a yeast fermentation product.

29. The method of any one of claims 2 to 28, wherein the glutamate is monosodium glutamate.

30. The method of any one of claims 2 to 28, wherein the glutamate is a glutamic acid, α-ketoglutarate, glutamine, L-glutamic acid or L-glutamine or a derivative thereof.

31. Use of the clay or clay blend of any one of claims 1-30 as a diet supplement.

32. The use according to claim 31, wherein the diet supplement is administered to shrimp.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for treating early mortality syndrome (EMS)/acute hepatopancreatic necrosis disease (AHPND) in an animal in need thereof comprising administering clay or a clay blend to the animal, thereby treating early mortality syndrome (EMS)/acute hepatopancreatic necrosis disease (AHPND).

2. The method of claim 1, wherein the clay blend comprises a clay, a glutamate and/or a yeast.

3. The method of claim 1, wherein the clay blend comprises a calcium montmorillonite clay.

4. The method of claim 1, wherein the early mortality syndrome (EMS)/acute hepatopancreatic necrosis disease (AHPND) is caused by a Vibrio species.

5. The method of claim 4, wherein the Vibrio species is Vibrio parahaemolyticus.

6. The method of claim 1, wherein the animal is a shrimp, fish, clam, crab, lobster, pig, cattle, sheep, goat, horse, or human.

7. The method of claim 1, wherein the clay or clay blend is administered as a diet supplement.

8. The method of claim 1, wherein the clay blend is about 50 to 90% w/w of the clay, about 0.01 to 20% w/w of the yeast and about 0.01 to 20% w/w of the glutamate.

9. The method of claim 8, wherein the clay blend is about 80% w/w of the clay, about 10% w/w of the yeast and about 10% w/w of the glutamate.

10. The method of claim 1, wherein the clay is a calcium montmorillonite clay.

11. The method of claim 1, wherein the clay is a sorbent mineral, a diatomaceous earth, a silicate, a zeolite, an attapulgite, or a combination thereof.

12. The method of claim 1, wherein the clay is heated to between about 100° C. to about 800° C.

13. The method of claim 1, wherein the clay is ground or exfoliated to a particle size of about 10 nanometers to 50 microns.

14. The method of claim 1, wherein the clay further comprises a surfactant.

15. The method of claim 14, wherein the surfactant is a soil surfactant or a surfactant containing alkoxylated polyols, humectants, alkylpolyglucoside esters, and polycarboxylates, sodium salts or a combination of soil surfactants and humectant complexes.

16. The method of claim 2, wherein the yeast is a Pichia yeast, a citric acid press cake, a yeast fermentation product or a yeast component.

17. The method of claim 16, wherein the yeast is a Pichia guilliermondii yeast.

18. The method of claim 16, wherein the yeast component is a yeast mannan, a yeast cell wall, a mannan oligosaccharide, a beta glucan, a fiber, a carbohydrate source, a prebiotic, or a combination thereof.

19. The method of claim 16, wherein the yeast component is isolated from grain, mushroom and/or bacteria.

20. The method of claim 2, wherein the glutamate is monosodium glutamate.

21. The method of claim 2, wherein the glutamate is a glutamic acid, α-ketoglutarate, glutamine, L-glutamic acid or L-glutamine or a derivative thereof.
